(19) United States
(12) Patent Application Publication
(10) Pub. No.: US 2006/0041261 A1
(43) Pub. Date: Feb. 23, 2006

(54) Apparatus and method for attaching connective tissue to bone

(76) Inventor: Thomas P. Osypka, Palm Harbor, FL (US)

Correspondence Address: EDWARDS & ANGELL, LLP, P.O. BOX 55874, BOSTON, MA, 02205, US

(21) Appl. No.: 11/128,762

(22) Filed: May 13, 2005

Related U.S. Application Data
(60) Provisional application No. 60/602,226, filed on Aug. 17, 2004.

(51) Int. Cl. A61B 17/58

(52) U.S. Cl. 606/73

(57) ABSTRACT

Disclosed is an apparatus for attaching tissue to bone. The apparatus includes a bone anchor having a distal anchoring portion for implantation in bone and a proximal reception portion that receives a fixation member. The fixation member has a distal engagement portion for releasably engaging the proximal reception portion of the bone anchor. A support flange included with the fixation member proximal to the distal engagement portion selectively compresses the tissue to be attached to the bone. In a preferred embodiment, the apparatus further includes an intermediate support member dimensioned and configured for placement between the proximal reception portion of the bone anchor and the support flange of the fixation member. Also disclosed is a method for attaching tissue to bone utilizing said apparatus.

### CROSS-REFERENCE TO RELATED APPLICATION

The subject application claims the benefit of commonly-owned, co-pending U.S. Provisional Patent Application Ser. No. 60/602,226, filed Aug. 17, 2004, the disclosure of which is herein incorporated by reference.

### BACKGROUND OF THE INVENTION

1. Field of the Invention

The present invention relates generally to methods and apparatus for attaching soft tissue to bone, and more particularly, to anchors and methods for securing connective tissue, such as ligaments or tendons, to bone. The present invention has particular application to arthroscopic surgical techniques, such as reattaching the rotator cuff to the humeral head in order to repair the rotator cuff.

2. Background of the Related Art

It is a common problem for tendons and other soft, connective tissues to separate from associated bone, either through tearing of the tissue itself or detachment of the tissue from the bone. One common example of this problem is the “rotator cuff” tear, wherein the supraspinatus tendon separates from the humerus, causing pain and an inability to elevate and externally rotate the arm. This condition can occur abruptly if the shoulder is subjected to gross trauma, although it more commonly results from the steady growth of initially small lesions (especially in older patients).

Once a rotator cuff is completely separated from the bone, repair typically requires a surgical procedure. In most cases (almost 99%), such surgery is “open”, meaning it is performed through a relatively large incision. These open surgical procedures are commonly classified as either a “classic open” procedure or a “mini-open” procedure, depending on the size of the incision and the details of the procedure. More recently, arthroscopic procedures have been developed for rotator cuff repair, allowing for a reduced incision size. All of these techniques are described and compared below.

The classic open technique begins with a large external incision and complete detachment of the deltoid muscle from the acromion, thereby exposing the underlying rotator cuff. The cuff is then debrided both to create a reasonable edge approximation and to ensure that viable tissue is used for subsequent reattachment to the humerus. In addition, the humeral head is abraded at the proposed cuff reattachment point, allowing the cuff to be reattached to a raw bone surface, a factor known to enhance the rate of healing. A series of small diameter holes, referred to as “transosseous tunnels”, are punched laterally through the bone, thereby extending from the abraded or notched surface to a point about 2 to 3 cm away on the outside surface of the greater tuberosity. Finally, the cuff is sutured and secured to the bone by pulling the suture ends through the transosseous tunnels and tying them together, thereby using the bone between two adjacent tunnels as a bridge. It is preferable to suture the cuff before tying the suture to the bone, as this allows the sutured ligament to be pulled towards the bone until the proper tension is achieved, at which point it can be fixed (a process known as “approximating the cuff to the bone”). After cuff reattachment is complete, the deltoid muscle is surgically reattached to the acromion.

Due to the use of a large incision and the need to detach the deltoid muscle, the classic open technique inflicts significant trauma on the deltoid and the surrounding tissues. This leads to an extended rehabilitation period, typically lasting from nine to 12 months. Further, the damage sustained by the deltoid necessitates postoperative deltoid protection, retarding rehabilitation and potentially resulting in residual weakness.

The mini-open technique differs from the classic approach by gaining access through a smaller incision and splitting, rather than detaching, the deltoid to expose the rotator cuff. As with the classic approach, the cuff is then debrided and the humeral head is abraded. The cuff is then sutured and attached to the humeral head using either transosseous tunnels or “bone anchors”. Bone anchors are devices that can be affixed to bone and provide structures to which sutures can be secured. Finally, the split deltoid is surgically repaired, completing the process. (Additionally, this procedure is typically performed in conjunction with arthroscopic acromial decompression.)

Because the mini-open technique utilizes a smaller incision and inflicts less trauma than the classic open approach, it is the more popular choice; presently, a majority of all surgical repair procedures are of the mini-open variety. However, despite its associated advancements, the mini-open technique, like the classic open, involves a great deal of patient discomfort, mainly owing to the relatively large skin incision and significant deltoid manipulation involved. Further, the typical recovery time of approximately four months to more than one year, while being reduced with respect to the classic open approach, is still quite lengthy.

While open surgical techniques represent the current standard of care for rotator cuff repair, the persistent problems that accompany these procedures have led to the development of less invasive arthroscopic rotator cuff repair techniques. Arthroscopic repair proceeds by inserting a narrow trocar portal successively through a small skin incision and the deltoid. Surgery is then performed entirely through the portal (using instruments customized for that purpose), thereby causing minimal deltoid disruption. A video camera is passed through the portal to the surgical site, allowing remote visualization while performing the procedure. The rotator cuff is sutured intracorporeally, and bone anchors are driven into bone for receiving the sutures. It should be noted that, unlike in open surgery, bone anchors are an essential component of arthroscopic rotator cuff repair, as it is not feasible to form transosseous tunnels arthroscopically.

Early results in the use of arthroscopic techniques are encouraging, with a substantial reductions in both patient recovery time and discomfort, improved infection rates, and better cosmesis. However, despite these advantages, arthroscopy is used in less than 1% of all repairs, and is still considered “investigational” in nature. This low frequency of use is due to two significant limitations of the arthroscopic procedures: the significant technical complexity involved in performing the procedure and the deficiencies in commonly available bone anchors. Both of these limitations are discussed below.

Several aspects of the conventional arthroscopic rotator cuff repair require an inordinately high physician skill level. First, intracorporeal suturing of soft tissues while working through a trocar under endoscopic visualization is clumsy and time consuming, and allows only the simplest suture stitch patterns to be utilized. Second, intracorporeal knot tying, necessary to secure the sutures to bone, is exceptionally challenging. Extracorporeal knot tying is somewhat less difficult, but the ultimate tightness of the knots is difficult to judge, and the tension cannot be adjusted later. These technical difficulties surpass those experienced in performing open surgery and contribute to the lack of use of arthroscopic rotator cuff repair.

Aside from the technical complexity involved with arthroscopic surgery, commonly available bone anchors have several inherent problems. A typical bone anchor includes an annular structure through which a suture can be threaded (the “eyelet”; similar to the eye of a needle) and a threaded portion that allows the bone anchor to be screwed into a bone (the “anchoring portion”). In practice, both features have proven problematic.

Eyelets serve as the lone structure securing the rotator cuff to bone. As such, in use, eyelets are required to support all loads applied to the rotator cuff. Given the necessarily small size of the eyelet, the loads experienced by the rotator cuff during normal shoulder use can result in high stresses in the eyelet, possibly leading to failure. Eyelet failure is a commonly seen problem, and is a concern for virtually all bone anchors available today.

The anchoring portion of a bone anchor often has a screw-like shape, allowing it to be screwed into bone. This securing method, while generally well-known, presents special challenges when used in bone. Specifically, existing bone anchor screws tend to loosen over time, an exceptionally deleterious phenomenon in light of the fact that retightening, if at all possible, requires another surgical procedure.

The problems associated with arthroscopic rotator cuff repair in general and those associated with bone anchors specifically have led to the development of both novel repair procedures and novel devices. These developments, discussed below, have alleviated some of the above-described problems. However, as will be seen, many challenges still remain.

Relatively recently, several non-screw based anchoring portions for bone anchors have been developed. One approach utilizes the difference in stiffness between cancellous bone (the soft and somewhat vascular interior of the bone) and the surrounding cortical bone (the bone's stiff, dense, outer shell). The anchor is designed with a large aspect ratio between a long and short axis. To secure the anchor to bone, a high aspect ratio hole is drilled in the cortical bone, the hole being sufficiently large to accommodate the anchor when the long axis of the anchor is aligned with the long axis of the hole, but not when these axes are misaligned. The anchor is inserted through the hole into the cancellous bone and rotated 90°, its long axis thereby disposed perpendicularly to the axis of the hole. Subsequent forces urging the anchor out of the hole cause the anchor to be seated up against the inside surface of the cortical bone, thus providing resistance to pull-out.

Another non-screw based anchoring portion uses a “pop rivet” approach. A hole is formed in the cortical bone, into which a split shaft is inserted. The split shaft is hollow and defines a lumen, and has a tapered plug extending from inside the lumen out through the top of the shaft. Once the shaft is inserted into the hole, the plug is driven further into the lumen, its tapered shape causing the split shaft to laterally expand. This expansion ostensibly locks the device into the bone.

Both of the previous anchoring portion designs potentially solve the problem of bone anchor loosening. However, these anchors are more complicated to manufacture than the simple screw, and neither concept addresses the aforementioned problem of eyelet failure. More recent bone anchor concepts, in addition to moving away from screw-like anchoring portions, have avoided use of eyelets altogether.

An example of an approach that eliminates the need for eyelets is disclosed in U.S. Pat. No. 5,324,308 to Pierce. In this patent, there is disclosed a suture anchor that incorporates a proximal and distal wedge component having inclined mating faces. The distal wedge component has two suture thread holes at its base through which a length of suture may be threaded. The assembly may be placed in a drilled hole in the bone, and when tension is placed on the suture, the distal wedge block is caused to ride up against the proximal wedge block, expanding the projected area within the drilled hole, and locking the anchor into the bone. The Pierce approach, while successful in eliminating both the eyelet and the screw-like anchoring portion, has several drawbacks, including the inability to suture the soft tissue prior to anchoring the suture to bone to allow approximating the soft tissue to bone, and, the use of a relatively complicated structure. Further, the problem of knot tying still remains.

U.S. Pat. No. 5,782,863 to Bartlett discloses a suture anchor including bone attachment, which simply comprises a conical suture anchor having an anchor bore through which a length of suture is threaded. The anchor is inserted into a bore within a portion of bone using an insertion tool having a shape memory insertion end. As the anchor is inserted, because of its conical shape, it will re-orient itself by rotating in order to fit into the bore, bending the end of the insertion tool. However, once the proximal edge of the bone anchor enters cancellous bone, the shape memory insertion end of the insertion tool will begin resuming its natural straight orientation, thus rotating the anchor back into its original orientation. The corners of the conical body thus protrude into the soft cancellous bone, and the anchor body is prevented from exiting proximally from the bone bore through the hard cortical bone. The insertion tool is then removed.

The approach of the Bartlett patent, as with the Pierce patent, while innovative, is disadvantageous to the extent that it involves the use of a unique and complex insertion tool that can be difficult to deploy. It also does not permit approximating the soft tissue to bone at the conclusion of the suturing procedure. Additionally, in preferred embodiments, the suture is knotted to the anchor, a known disadvantage. Finally, Bartlett requires the challenging step of knot tying to fix the tissue to the bone.

Yet another prior art approach is disclosed in U.S. Pat. No. 5,961,538 to Pedlick et al. This patent describes a wedge shaped suture anchor system for anchoring a length of suture within a bore in a bone. The system comprises an anchor body having an offset suture opening for receiving the length of suture therethrough, and for creating an imbalance in the rotation of the device as it is inserted. A shaft portion is utilized to insert the wedge-shaped anchor body into the bone bore. Once the anchor body is in cancellous bone, the shaft is pulled proximally to cause the anchor body to rotate, thereby engaging the corners of the anchor body with the cancellous bone. The shaft then becomes separated from the anchor body, leaving the anchor body in place within the bone.

As with some of the other concepts already discussed, the Pedlick et al. approach requires that the suture be attached to the soft tissue of interest only after it is already anchored within the bone. Consequently, there is no opportunity to optimally approximate the soft tissue to the bone upon completion of the surgical procedure. Additionally, the approach is complex and limited in flexibility, since the suture is directly engaged with the bone anchoring body. There is also the possibility that the bone anchoring body will not sufficiently rotate to firmly become engaged with the cancellous bone before the insertion tool breaks away from the anchor body, in which case it will be impossible to properly anchor the suture. Finally, the problem of knot tying remains.

U.S. Pat. No. 6,056,773 to Bonutti discloses a suture anchoring system which is somewhat similar to that disclosed by Pedlick et al. A cylindrical suture anchor body is provided which is insertable into a bone bore, using a pusher member that pushes distally on the anchor body from a proximal direction. As the anchor body proceeds into the bone bore, below the cortical bone surface, the suture extending through the lumen of the anchor body applies a proximal tensile force on the anchor body, to cause the anchor body to rotate relative to the pusher member, thereby anchoring the anchor body in cancellous bone. Of course, this system has similar disadvantages to those of the Pedlick et al. system.

All of the previously described concepts address the problems associated with eyelets and screw-like anchoring portions. However, none in any way address the problem of intracorporeal knot tying for suture fixation. The difficulties associated with tying knots in an endoscopic environment are well known, and there have been attempts to simplify the process of suture fixation. One such approach is disclosed in U.S. Pat. No. 5,383,905 to Golds et al. The Golds et al. patent describes a device for securing a suture loop that includes a bead member having a longitudinal bore and an anchor member adapted to be slidably inserted within the bore of the bead member. The anchor member includes at least two axial compressible sections that define a passageway to receive two end portions of a suture loop. The axial sections collapse radially inwardly upon insertion of the anchor member within the bore of the bead member to securely wedge the suture end portions received within the passageway.

Although the Golds et al. patent approach utilizes a wedge-shaped member to lock the sutures in place, the suture legs pass through the bore of the bead only one time, in a proximal to distal direction, and are locked by the collapsing of the wedge, which creates an interference on the longitudinal bore of the anchor member. As such, the design is primarily suited for locking a suture loop, such as is used for ligation or approximation of soft tissues, rather than fixing sutures to bone. The Golds et al. patent, used in conjunction with some of the bone anchor ideas previously presented, would provide a solution to many of the technical challenges involved in arthroscopic rotator cuff repair. However, such a solution would make for an expensive combination of complicated parts, and would still be prone to all of the previously highlighted problems related to the bone anchors.

Several ideas have been presented that are claimed to provide complete solutions for fixing sutures to bone. For example, a disclosure that incorporates bone attachment and eliminates knot tying is set forth in U.S. Pat. No. 5,702,397 to Goble et al. One embodiment, in particular, is shown in FIG. 23 of that patent and includes a bone anchor that has a threaded body with an inner cavity. The cavity is open to one end of the threaded body, and joins two lumens that run out to the other end of the threaded body. Within the cavity is disposed a gear, journaled on an axle. A length of suture is threaded through one lumen, around the gear, and out through the other lumen. A ball is disposed within the cavity to ride against a tapered race and ostensibly lock the suture in place. The disclosure purports that tension in the suture would lock the ball into the race and secure the suture. However, the construct shown is complicated, and does not appear adequate for reliably fixating the suture. Further, its threaded anchoring portion is potentially vulnerable to loosening over time.

Another prior art approach is described in U.S. Pat. No. 5,405,359 to Pierce. In this system, a toggle wedge is comprised of a two-piece structure comprising a top portion characterized by the presence of a barbed tip and a bottom portion. The suturing material extends through apertures in each of the two toggle portions, and is maintained in position by means of a knot disposed in the suture at a lower edge of the bottom toggle portion. To anchor the suture into adjacent soft tissue, the two toggle portions are rotated relative to one another, as exemplified in FIG. 33. The disclosure states that the device could be used to anchor a suture in bone, as well as soft tissue, if two embodiments are utilized in tandem; however, it is not clear from the disclosure how such tandem use might take place. Further, the system is disadvantageous in that it is complex and difficult to manipulate.

Another approach that includes attachment to both soft tissue and bone without knot tying is described in U.S. Pat. No. 5,584,835 to Greenfield. In this patent, a two-part device for attaching soft tissue to bone is shown. A bone anchor portion is screwed into a hole in the bone, and is disposed to accept a plug that has been adapted to receive sutures. In one embodiment, the suture plug is configured so that when it is forced into its receptacle in the bone anchor portion, sutures that have been passed through an eyelet in the plug are trapped by friction between the wall of the anchor portion and the body of the plug.

Although this approach eliminates the need for knots in the attachment of sutures to bone, it creates new obstacles to properly setting the tension in the sutures. The user is required to first pull the sutures through the plug until the appropriate tension is achieved, and then to force the plug further into the bone anchor portion. This action increases the tension in the sutures, and may cause garroting of the soft tissues or failure of the sutures. In addition, the minimal surface area provided for gripping the sutures between the plug and the wall of the anchor portion will accelerate abrasion of the suture such that its ability to carry load will be greatly compromised.

Finally, in U.S. Pat. No. 6,770,076 to Foerster, a method and apparatus for attaching connective tissues to bone using a knotless suture-anchoring device is described. This apparatus is manufactured by Opus Medical and is currently used in the field. Although the system provides what can be described as a simplified approach for fixating a suture to both a bone screw and connective tissue, the device itself is complicated to use and expensive to manufacture.

The preceding discussion has illustrated the difficulties attendant to suturing soft tissue, especially in an arthroscopic procedure. However, even in cases where the procedure is successfully completed and the associated devices (e.g. bone anchor) perform reliably, problems can ensue due to failure of the tissue being sutured. Recent studies have found re-tear rates as high as 70-90% following arthroscopic rotator cuff repair.¹ The study found that, while arthroscopic and open surgery resulted in the same level of incidence of “small” (<3 cm) tears, the frequency of “large” (>3 cm) post-operative tears was significantly higher following arthroscopy. This re-tearing is presumably due to the small surface area between the suture and the ligament that is required to carry the load exerted on the rotator cuff.
¹Bishop et al., “Cuff Integrity Following Arthroscopic Versus Open Rotator Cuff Repair: A Prospective Study”, American Orthopedic Society for Sports Medicine (AOSSM) Specialty Day, Mar. 13, 2004.

The preceding discussion has generally shown that the existing methods for suturing rotator cuffs and the bone anchors used in these procedures are not optimal. Methods of securing soft tissue to bone other than suturing are known in the prior art, and include staples, tacks, and the like. However, these methods are not presently considered to be feasible for shoulder repair procedures, due to the possibility that these items could fall out and cause injury during movement. As such, physicians are reluctant to leave anything but a suture in the capsule area of the shoulder, and when something other than a suture must be used, the attachment point often must be located at a less than ideal position. Further, both tacks and staples require a substantial hole in the soft tissue, and make it difficult for the surgeon to precisely locate the soft tissue relative to the bone.

What is needed, therefore, is a new method for re-attaching soft tissue to bone that is simple and inexpensive, but at the same time reliable. The method ideally should avoid the requirement for the surgeon to tie a knot to fix the tissue to the bone and should allow the tension in the tissue to be adjusted and possibly measured. Finally, the new approach should address the problem of tissue re-tearing that plague existing procedures.

### SUMMARY OF THE INVENTION

The subject invention is directed to an apparatus for attaching tissue to bone. The apparatus includes a bone anchor having a distal anchoring portion for implantation in bone and a proximal reception portion that receives a fixation member. The fixation member has a distal engagement portion for releasably engaging the proximal reception portion of the bone anchor. A support flange included with the fixation member proximal to the distal engagement portion selectively compresses the tissue to be attached to the bone.

In a preferred embodiment, the apparatus further includes at least one intermediate support member dimensioned and configured for placement between the proximal reception portion of the bone anchor and the support flange of the fixation member. This placement can be between connective tissue and the reception portion of the bone anchor, or, between connective tissue and the support flange of the fixation member. In another preferred embodiment, the apparatus includes a first and a second intermediate support member, the first adapted for placement between connective tissue and the support flange of the fixation member and the second adapted for placement between connective tissue and the reception portion of the bone anchor. In yet another preferred embodiment, the apparatus includes a support member that is a generally rectangular and planar and is includes a plurality of apertures for receiving a corresponding number of fixation members.

The subject invention is also directed to a method for attaching tissue to bone utilizing the above-described apparatus. The bone anchor is secured in bone. The distal engagement portion of the fixation member is extended through the tissue to be attached to the bone and engages with the proximal reception portion of the bone anchor. This secures the tissue under repair between the support flange of the fixation member and the proximal reception portion of the bone anchor. In an alternative preferred method, at least one intermediate support member is positioned between the proximal reception portion of the bone anchor and the support flange of the fixation member, either between the tissue and flange or between the tissue and bone anchor. In another alternative preferred method, at least two intermediate support members are positioned both between the tissue and flange and between the tissue and bone anchor, respectively.

The above-described apparatus and method provide a new process for attaching tissue to bone. The process is simple and inexpensive due to the use of uncomplicated parts, and avoids the troublesome requirement for the surgeon to tie a knot to fix the tissue to the bone. Further, the surgeon can adjust the tension in the tissue during the procedure by inserting the fixation member deeper into the bone anchor. The process is also reliable, due to both the simplicity of implementation and the use of intermediate support members and flanges to distribute stresses on the tissue. This last point addresses the problem of tissue re-tearing that plagues existing procedures.

### BRIEF DESCRIPTION OF THE DRAWINGS

So that those having ordinary skill in the art to which the subject invention appertains will more readily understand how to make and use the anchor assembly of the subject invention, preferred embodiments thereof will be described in detail hereinbelow with reference to the drawings, wherein:

FIG. 1 is a side elevational view of a human shoulder with a torn rotator cuff, showing the supraspinatus tendon detached from the humeral head;

FIG. 2 is a cross-sectional view of the shoulder of FIG. 1 after repair of the rotator cuff, the repair being completed utilizing a tissue reattachment apparatus in accordance with the present invention;

FIG. 3 is an exploded perspective view of a first embodiment of the connective apparatus of the subject invention, which includes a fixation member adapted and configured for threaded engagement with a bone anchor;

FIG. 4 is a perspective view of the fixation member of FIG. 3;

FIG. 5 is a side view of the fixation member of FIG. 3;

FIG. 6 is a side view of the bone anchor of FIG. 3, with a portion of the bone anchor removed to reveal the threaded internal surface of the bone anchor;

FIG. 7 is an exploded perspective view of an alternative embodiment of the connective apparatus of the subject invention, in which the fixation member is inserted through an intermediate support member before engaging the bone anchor;

FIG. 8 is a cross-sectional view of the intermediate support member of FIG. 7, the view taken along line 8-8 in FIG. 7;

FIG. 9 is a cross-sectional view of the connective apparatus of FIG. 8 while being utilized in a patient, showing the intermediate support member positioned between the flange of the fixation member and the tissue being repaired;

FIG. 10 is a perspective view of an alternative embodiment of the intermediate support member in which the intermediate support member has a square perimeter;

FIG. 11 is a cross-sectional view of the connective apparatus of FIG. 8 while being utilized in a patient, showing the intermediate support member positioned between the bone anchor and the tissue being repaired;

FIG. 12 is a cross-sectional view of an alternative embodiment of the connective apparatus of the present invention while being utilized in a patient, including two intermediate support members positioned between the flange and tissue and between the tissue and bone anchor, respectively;

FIG. 13 is an exploded perspective view of another embodiment of the connective apparatus of the subject invention, which includes a fixation member adapted and configured for interlocking engagement with a bone anchor;

FIG. 14 is a perspective view of the fixation member of FIG. 13, showing the key at distal engagement portion of the fixation member;

FIG. 15 is a side view of the fixation member of FIG. 13;

FIG. 16 is a side view of the bone anchor of FIG. 13, showing the inverted T-shaped keyway formed in the proximal reception portion for engaging a complementary-shaped key on the fixation member;

FIG. 17 is an exploded perspective view of another embodiment of the connective apparatus of the subject invention, showing the inclusion in the head of the fixation member a central hexagonal recess;

FIG. 18 is an exploded perspective view of still another embodiment of the connective apparatus of the subject invention, showing an intermediate support member adapted and configured to receive a plurality of fixation members;

FIG. 19 is a cross-sectional view of the embodiment depicted in FIG. 18 while being utilized in a patient;

FIG. 20 is a perspective view of another preferred embodiment of the present invention, showing a mode of securing tissue to bone that is reversed from that of FIG. 3;

FIG. 21 is a perspective view of another preferred embodiment of the present invention, showing an intermediate support member 630 having a main area and an elongated strap extending from main area;

FIGS. 22-25 are perspective views illustrating the process of incrementally pulling tissue to bone using the structure of FIG. 21.

### DETAILED DESCRIPTION OF PREFERRED EMBODIMENTS

The present invention solves the problems outlined above by providing innovative apparatus and techniques for connecting tissues to bone. The connective techniques permit a sutureless attachment, eliminating the need for placing suture wires and tying knots, both of which are particularly arduous and technically demanding tasks when performed arthroscopically.

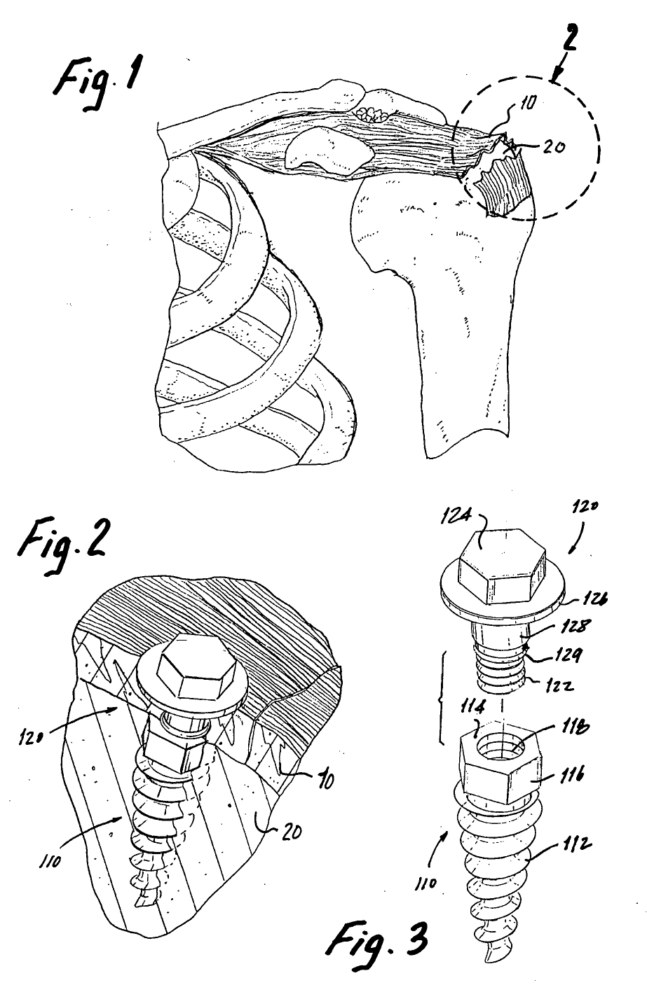

Referring now to the accompanying drawings wherein like reference numerals identify similar structural features of the present invention, there is illustrated in FIG. 1 a variety of rotator cuff tear in which the supraspinatus tendon 10 has separated from the associated humeral head 20. FIG. 2 illustrates tendon 10 after it has been repaired through the use of an apparatus for attaching tissue to bone constructed in accordance with the present invention and designated generally by reference numeral 100.

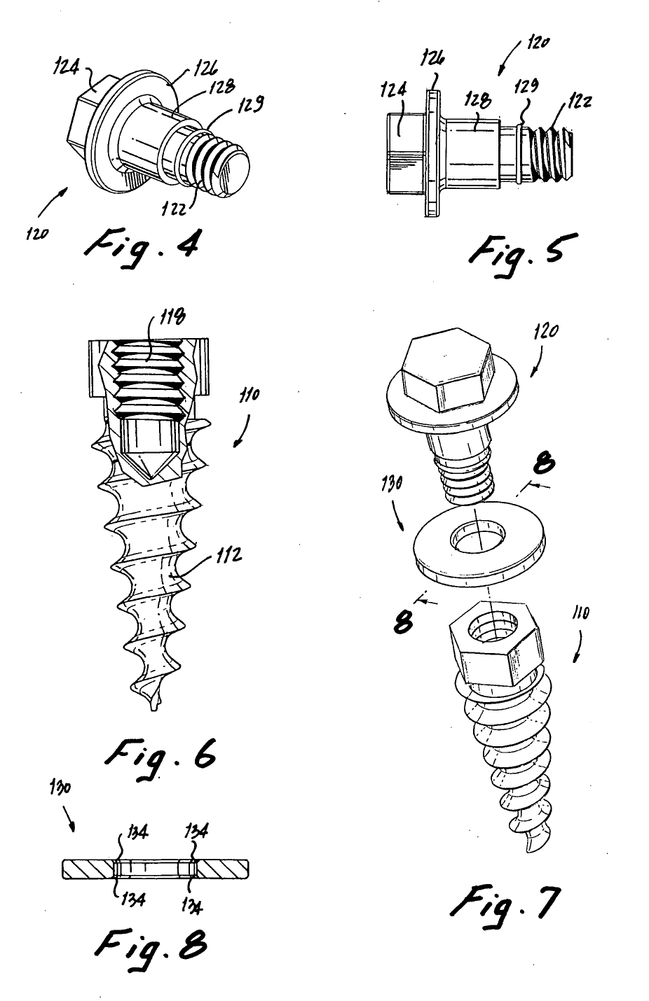

Referring to FIGS. 2-6, apparatus 100 includes an elongated bone anchor 110 and a fixation member 120. Bone anchor 110 has a distal anchoring portion 112 for implantation in bone and a proximal reception portion 114 for receiving fixation member 120. Anchoring portion 112 is threaded, and reception portion 114 has a hexagonal outer surface 116 that allows anchor 110 to be screwed into bone 20 using an appropriate tool (e.g. a wrench). This method of attaching anchor 110 to bone 20 is both simple and inexpensive.

A variety of thread dimensions for anchoring portion 112 are compatible with the present invention, several of which are commonly used in prior art bone anchors. In a preferred embodiment, the anchoring portion 112 is tapered at an angle of about 8°, and the threads have a pitch of about 1.25 mm and a thread depth of about 0.54 mm. These dimensions of anchoring portion 112 are chosen to facilitate secure placement of the anchor 110 and avoid anchor loosening over time.

While the anchoring portion 112 secures the anchor 110 in bone 20, concave reception portion 114 releasably engages the fixation member 120. The internal surface 118 of the reception portion 114 and the distal engagement portion 122 of the fixation member 120 are cooperatively threaded. The fixation member 120 is thus screwed into the anchor 110 using an appropriate driving tool to engage a hexagonal protrusion 124 included on the fixation member 120.

When using apparatus 100 to attach tissue to bone, as exemplified in FIG. 2, the bone anchor 110 is secured to bone 20 by screwing the threaded anchoring portion 112 into the bone 20. Engagement portion 122 of the fixation member 120 penetrates the tissue 10 and is screwed into reception portion 114 of the anchor 110. The fixation member 120 includes a support flange 126 that serves to compress and grip the tissue 10; the tissue is held in place both by interacting with the body 128 of the fixation member 120 and by compression of the flange 126. Both body 128 and flange 126 tend to provide a smooth surface for contacting tissue, reducing the trauma inflicted by the securing procedure. If need be, the fixation member 120 can be unscrewed from the anchor 110, and the releasable nature of this connection allows the tension in tissue 10 to be adjusted and optimized during attachment. In a preferred embodiment, an O-ring 129 is placed around body 128 to seal with inner surface 118 and prevent material from entering concave reception portion 114.

As is clear from the above description, the proposed method for attaching tissue to bone eliminates the challenging steps of suture placement and knot tying. Further, the method utilizes simple tools and procedural steps, making the method both inexpensive and time saving.

In an alternative preferred procedure for utilizing apparatus 100, engagement portion 122 of the fixation member 120 penetrates the tissue 10 and is screwed into reception portion 114 of bone anchor 110. Bone anchor 110 is secured to bone 20 by screwing the threaded anchoring portion 112 into the bone 20. The tension in tissue 10 is adjusted and optimized during attachment by varying the depth to which bone anchor 110 is driven into bone 20.

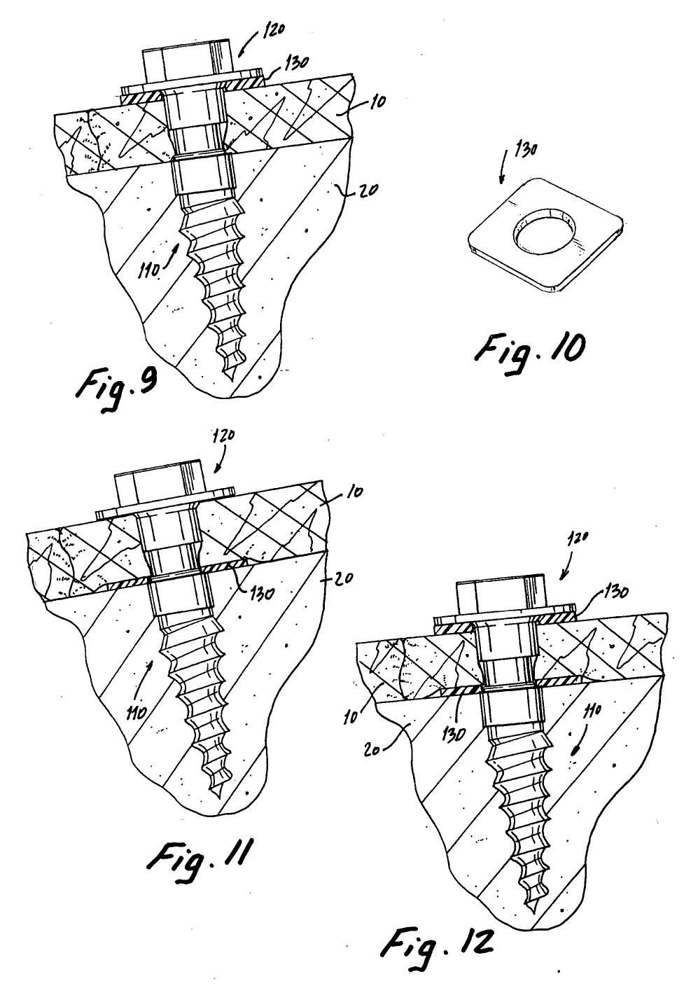

In some cases, as shown in FIGS. 7-9, it may be desirable to include an intermediate support member 130. In a preferred embodiment, the intermediate support member 130 is planar and defines an aperture 132 sized to allow the engagement portion 122 and body 128 of the fixation member 120, but not flange 126, to pass through. When using an intermediate support member 130 in the securing of tissue, the engagement portion 122 of the fixation member 120 successively is inserted through the aperture 132 and pierces the tissue 10, finally engaging the reception portion 114 of anchor 110. In this configuration, flange 126 abuts support member 130 and forces support member 130 into contact with the tissue 10. Through this action, the compressive load of the flange 126 is transmitted to tissue 10 through support member 130. It should be noted that, while the intermediate support member 130 is represented in FIG. 8 as round and annular, other shapes are also possible. For example, as shown in FIG. 10, the support member can be substantially square. Other shapes, such as a C-shape, are also possible.

The intermediate support member 130 can be wider than, narrower than, or coextensive with the support flange 126. In cases where the support member 130 is more extensive than the support flange 126, the support member 130 acts to spread the compressive force exerted on tissue 10 by the fixation member 120 over a wider area than would the flange 126 alone. This redistribution of the forces reduces stress in the tissue 10 and alleviates the significant problem of tissue re-tearing that has been seen for existing repair procedures. This stress reduction is also helpful in cases where the tissue being secured is exceptionally soft or fragile. Further, the support member 130 has rounded edges 134 to eliminate stress concentrations.

Other configurations of the intermediate support member 130 may be useful. For example, referring to FIG. 11, in order to prevent rupture of the connective tissue and facilitate healing, it may be useful to place the intermediate support member 130 between the tissue 10 and the anchor 110, by inserting the fixation member 130 first through the tissue 10 and then through the support member 130. In other applications, it may be helpful to include multiple intermediate support members 130, both between the tissue 10 and flange 126 and between the tissue 10 and anchor 110 (FIG. 12).

In preferred embodiments, bone anchor 110 and fixation member 120 are constructed of a biocompatible material. For example, a biocompatible implantable grade metal, such as titanium, stainless steel, MP35N® alloy (nickel-cobalt-chromium-molybdenum alloy), or the like can be used. Strong biocompatible plastics, like PEEK® (Polyether Ether Ketone) and other similar materials, are also appropriate. In some cases, a biodegradable material, such as a material formed from a mixture of glycolite and alactic acid, may be desirable.

The intermediate support member 130 is preferably constructed of an implantable grade silicone, thereby providing a compliant surface against which tissue may rest and further protecting soft or fragile tissues. In another preferred embodiment, support member 130 is formed of reinforced silicone or other more rigid material. The support member 130 can be reinforced, for example, with Dacron or metal mesh. Using a more rigid material reduces the ability of the support member 130 to conform to the bone and tissue, but is more effective in spreading the compressive force of the fixation member 120. Also, it is preferable that the support member 130 contains or is coated with a drug appropriate for use in such procedures and which gradually diffuses into connective tissue. The term “drug” as used herein is intended to mean any compound, which has a desired pharmacologic effect. For example, the drug can be an anti-inflammatory agent such as the steroid dexamethasone sodium phosphate or the like, or can be an anti-rejection agent, a painkiller, an anti-thrombolytic agent, or an anti-microbial or anti-bacterial or anti-viral agent, among others.

It should be noted that, while the apparatus has been described in accordance with rotator cuff repair, it is not limited to such procedures, but can be used generally to affix material to bone. Further, it should be clear that, while the above description has referred to the use of an individual apparatus to repair torn tissue, multiple apparatus can be used to fixate the same tissue. Finally, it should also be understood that the foregoing is only illustrative of exemplary and preferred embodiments, as well as principles of the subject invention, and that various modifications can be made by those skilled in the art without departing from the scope and spirit of the invention.

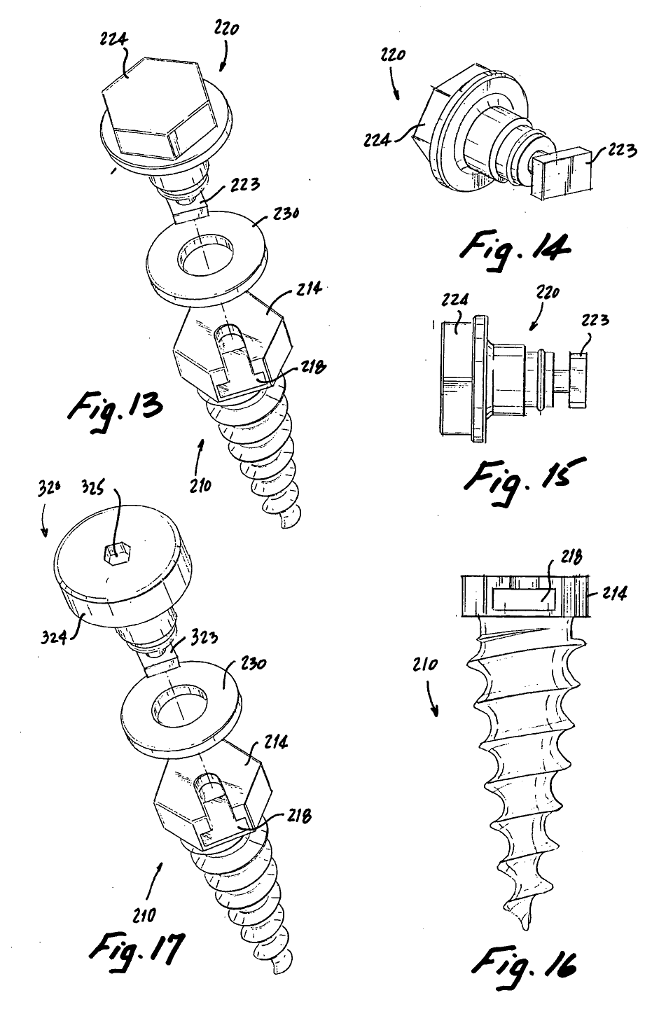

For example, FIGS. 13-16 illustrate an alternative preferred embodiment of the present invention. Bone anchor 210 includes an inverted T-shaped keyway 218 formed in the proximal reception portion 214, and fixation member 220 has a complementary-shaped key 223 at distal engagement portion 222. In a preferred embodiment, key 223 can be inserted into keyway 218 and fixation member 220 rotated with respect to anchor 210 to releasably interlock fixation member 220 and anchor 210. This can be accomplished, for example, by dimensioning both the key and keyway to have longer and shorter axes, such that when fixation member 220 is rotated with respect to anchor 210 and the long axes are misaligned, the interior surface of the reception potion creates an interference. Rotation is accomplished by applying an appropriate tool (e.g. a wrench) to the hexagonal protrusion 224 of fixation member 220. In another preferred embodiment, key 223 and keyway 218 can be formed so as to facilitate a snap-fit between the anchor 210 and the fixation member 220, removing the need to rotate fixation member 220 with respect to anchor 210. As with the previous embodiments, this preferred embodiment may or may not be used in conjunction with intermediate support member 230.

FIG. 17 illustrates another preferred embodiment of the present invention in which a bone anchor 310 and a fixation member 320 have the cooperative interlocking engagement illustrated in the previous embodiment and an alternative configuration for manipulating fixation member 320. In particular, head 324 of fixation screw 320 includes a central hexagonal recess 325 which may be manipulated and/or driven using a corresponding tool, such as a screwdriver or wrench.

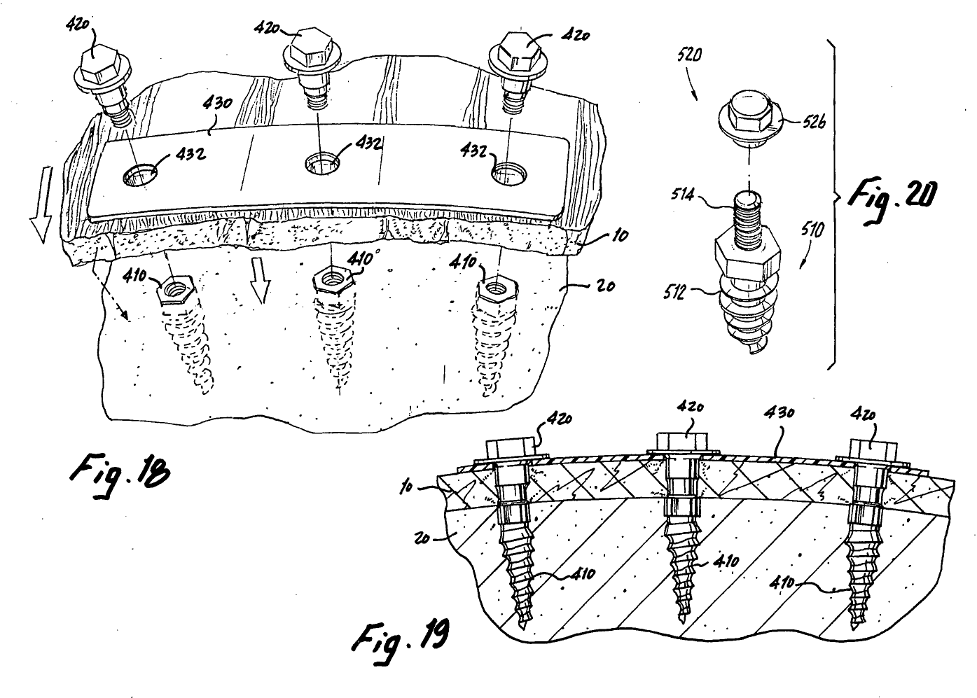

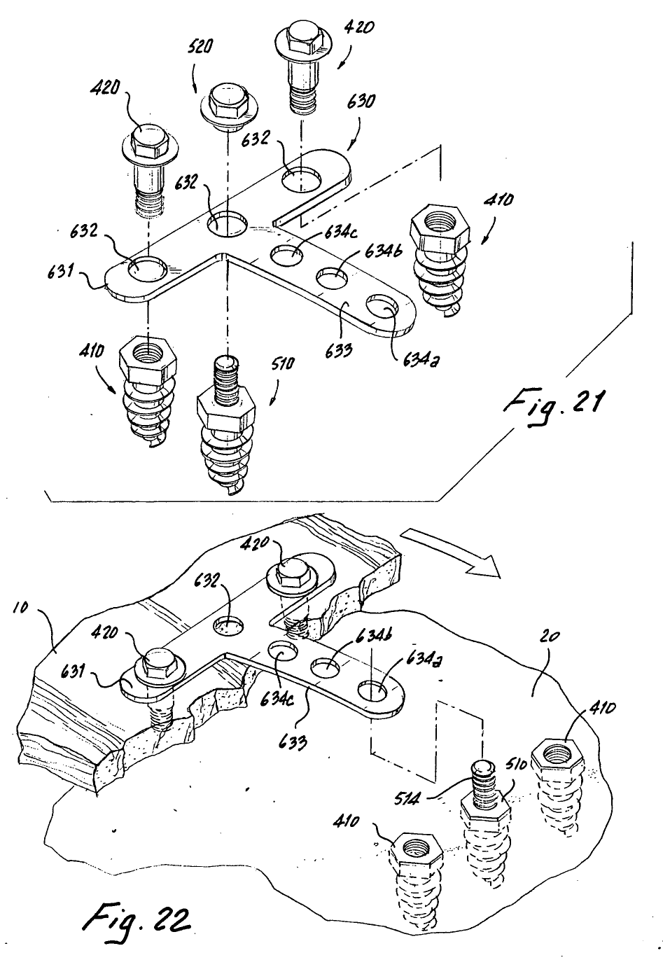

FIGS. 18 and 19 illustrate yet another preferred embodiment of the present invention. In this embodiment, intermediate support member 430 is generally rectangular and planar, and defines a plurality of apertures 432. A corresponding number of fixation members 420 are inserted through apertures 432 and then pierced through tissue 10. The fixation members 420 then secure to multiple bone anchors 410, which are implanted in bone 20. In this way, several fixation members 420 are used to secure the same general mass of tissue 10, and the compressive force exerted by the fixation members 420 on the tissue 10 can be distributed over the entire area between the fixation members 420 (i.e. the projected area of the intermediate support member 430).

FIG. 20 illustrates another preferred embodiment of the present invention. In this case, the mode of securing the tissue to bone is reversed from that shown in FIG. 3. Specifically, rather than utilizing a bone anchor 110 with a concave reception portion 114 that receives the fixation member 120 (as shown in FIG. 3), FIG. 20 shows a fixation cap 520 that engages with engagement portion 514 of bone anchor 510. The engagement can be completed, for example, by using a cooperative threading arrangement, or, by using an interlocking structure as previously described.

When employing the structure of FIG. 20, bone anchor 510 is secured to the bone 20 by screwing the threaded anchoring portion 512 into the bone 20. The tissue to be secured is pulled toward the bone until engagement portion 514 of the bone anchor 510 penetrates the tissue 10. Fixation cap 520 is then screwed onto engagement portion 514. The fixation cap 520 includes a support flange 526 that serves to compress and grip the tissue 10. If desired, one or more intermediate support members can be utilized in conjunction with the bone anchor 510 and fixation cap 520. Generally, this procedure allows the physician to perform the various aspects of the procedure without having his or her view obstructed by the tissue being repaired, a potentially desirable characteristic.

FIGS. 21-25 display another preferred embodiment of the subject invention. Here, intermediate support member 630 has a main area 631 and an elongated strap 633 extending from main area 631. The main area 631 defines a plurality of apertures 632, the apertures 632 generally linearly aligned. Strap 633 also defines a plurality of apertures 634a-c, the apertures 634a-c of the strap 633 being generally linearly aligned along a direction substantially perpendicular to the direction of alignment of the apertures 632 in the main area 631.

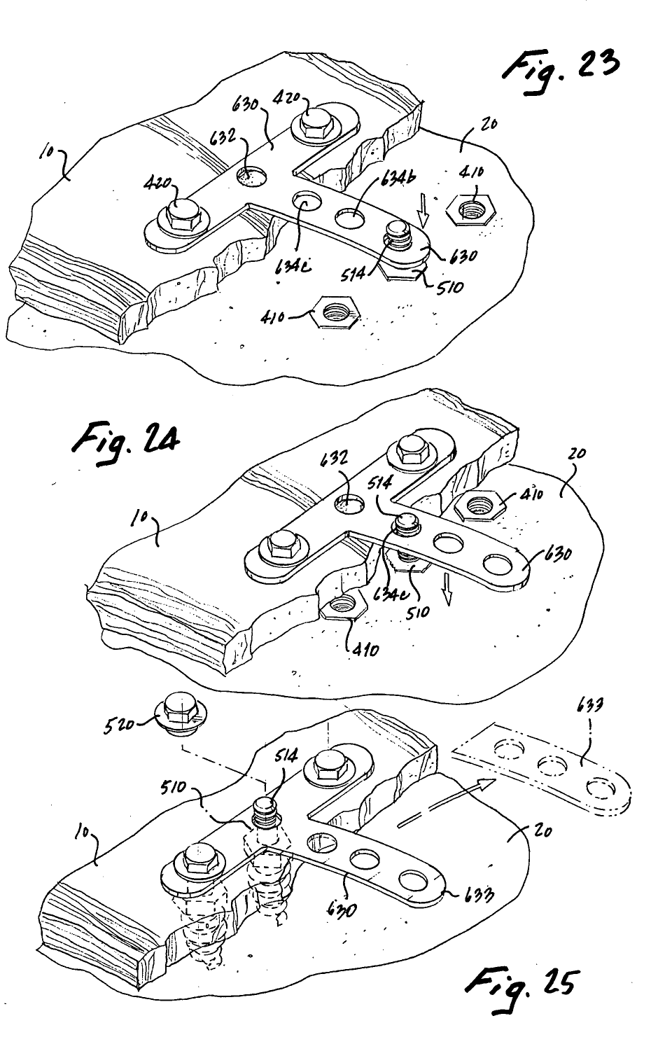

The intermediate support member of FIGS. 21-25 allows soft tissue 10 to be incrementally pulled toward, and then secured to, bone 20. When such a structure is being implemented, two different types of bone anchors are required. Bone anchors 410 and bone anchor 510 are implanted in bone 20. Fixation members 420, in number corresponding to the number of bone anchors 410, are inserted through apertures 632 and then pierced through tissue 10, thereby affixing the intermediate support member 630 to tissue 10. The physician then pulls the intermediate support member 630 to bring the tissue 10 toward the reattachment point on bone 20 (i.e., the intermediate support member 630 is pulled along the “attachment direction”). As the intermediate support member 630 is pulled, aperture 634a comes into registration with bone anchor 510, at which point, strap 633 is lowered to cause aperture 634a to encircle and engage engagement portion 514 of bone anchor 510; intermediate support member 630 is thus fixed at this location. (See FIG. 22.) Strap 633 can then be lifted off bone anchor 510 and pulled further along the attachment direction until the adjacent aperture 634b is aligned with bone anchor 510, at which point the strap 633 can be lowered onto bone anchor 510. This process can be repeated to secure strap 633 by aperture 634c (FIG. 24), and again until tissue 10 is correctly located for reattachment (FIG. 25). Fixation members 420 are aligned and engaged with mating bone anchors 410. Bone anchor 510 protrudes through one of the apertures 632 and is received by fixation cap 520. If desired, strap 633 can be removed post-attachment (FIG. 25).

This above described structure and tissue reattachment strategy allows the physician to incrementally pull the tissue to the correct location during reattachment. The ability to fix the tissue at intermediate locations is advantageous, as a large force is often required to pull the tissue into the proper position, leading to significant exertion on the part of the physician.

It should be noted that other designs for anchoring portion 112 besides a screw-like attachment are also compatible with the present invention. Specifically, many of the prior art bone attachment methods discussed in the background section are compatible with the present invention.

Therefore, the described embodiments should not be understood to limit the present invention in any way. Accordingly, the present disclosure embraces alternatives, modifications and variations of the present invention as fall within the spirit of the present disclosure and supplemental material appended hereto and incorporated by reference into the subject application.

What is claimed is:

1. Apparatus for attaching tissue to bone comprising:
a) an elongated bone anchor having a distal anchoring portion for implantation in bone and a proximal reception portion for receiving a fixation member; and
b) a fixation member having a distal engagement portion for releasably engaging the proximal reception portion of the bone anchor and a support flange proximal to the distal engagement portion for selectively compressing the tissue to be attached to the bone.

2. Apparatus as recited in claim 1, wherein the distal anchoring portion of the bone anchor includes means for securing the bone anchor to bone.

3. Apparatus as recited in claim 1, further comprising at least one intermediate support member dimensioned and configured for placement between the proximal reception portion of the bone anchor and the support flange of the fixation member.

4. Apparatus as recited in claim 3, wherein the at least one intermediate support member is adapted for placement between connective tissue and the reception portion of the bone anchor.

5. Apparatus as recited in claim 3, wherein the at least one intermediate support member is adapted for placement between connective tissue and the support flange of the fixation member.

6. Apparatus as recited in claim 3, including at least first and second intermediate support members, wherein the first intermediate support member is adapted for placement between connective tissue and the support flange of the fixation member and the second intermediate support member is adapted for placement between connective tissue and the reception portion of the bone anchor.

7. Apparatus as recited in claim 3, wherein the at least one support member is a generally annular member having a substantially planar configuration.

8. Apparatus as recited in claim 7, wherein the generally annular support member and the support flange of the fixation member are diametrically coextensive.

9. Apparatus as recited in claim 3, wherein the at least one support member has at least one aperture for receiving the engagement portion of the fixation member.

10. Apparatus as recited in claim 3, wherein the at least one support member is a generally rectangular member having a substantially planar configuration and a plurality of apertures for receiving a corresponding number of engagement portions of plural fixation members.

11. Apparatus as recited in claim 3, wherein the at least one support member is formed at least in part from a material having a medicament contained therein for gradual elution into connective tissue.

12. Apparatus as recited in claim 1, wherein the distal anchoring portion of the bone anchor is threaded.

13. Apparatus as recited in claim 12, wherein the distal anchoring portion of the bone anchor is tapered.

14. Apparatus as recited in claim 13, wherein the distal anchoring portion of the bone anchor has a taper angle of about 8°.

15. Apparatus as recited in claim 12, wherein the distal anchoring portion of the bone anchor has a thread pitch of about 1.25 mm.

16. Apparatus as recited in claim 12, wherein the distal anchoring portion of the bone anchor has a thread depth of about 0.54 mm.

17. Apparatus as recited in claim 1, wherein the proximal reception portion of the bone anchor has a hexagonal driving portion for cooperating with a driving tool.

18. Apparatus as recited in claim 1, wherein the fixation member has a hexagonal head portion proximal to the support flange for cooperating with a driving tool.

19. Apparatus as recited in claim 1, wherein the fixation member has a head portion proximal to the support flange with a hexagonal driving port formed therein for cooperating with a driving tool.

20. Apparatus as recited in claim 19, wherein a distal surface of the head portion defines the support flange.

21. Apparatus as recited in claim 1, wherein the distal engagement portion of the fixation member and the proximal reception portion of the bone anchor have a cooperative threading arrangement.

22. Apparatus as recited in claim 1, wherein the distal engagement portion of the fixation member and the proximal reception bore of the bone anchor have a cooperative interlocking configuration.

23. Apparatus as recited in claim 22, wherein the cooperative interlocking configuration includes an inverted T-shaped keyway formed in the proximal reception portion of bone anchor and a complementary shaped key formed on the distal engagement portion of the fixation member.

24. The apparatus as recited in claim 3, wherein the reception portion of the bone anchor includes a protrusion and the intermediate support member is affixed to the tissue by the fixation member, the intermediate support member including a strap that defines a plurality of apertures for receiving the protrusion of the bone anchor, thereby allowing the bone anchor to sequentially engage the plurality of apertures as the tissue is urged toward the bone.

25. Apparatus as recited in claim 3, wherein the at least one intermediate support member is composed of reinforced silicone.

26. A method for attaching tissue to bone comprising the steps of:
a) providing an elongated bone anchor having a distal anchoring portion for implantation in bone and a proximal reception portion for receiving a fixation member;
b) providing a fixation member having a distal engagement portion for engaging the proximal reception portion of the bone anchor and a support flange proximal to the engagement portion;
c) securing the bone anchor in bone;
d) extending the distal engagement portion of the fixation member through tissue to be attached to the bone; and
e) engaging the distal engagement portion of the fixation member with the proximal reception portion of the bone anchor, so as to secure the tissue between the support flange of the fixation member and the proximal reception portion of the bone anchor.

27. A method for attaching tissue to bone according to claim 26, further comprising the step of positioning at least one intermediate support member between the proximal reception portion of the bone anchor and the support flange of the fixation member.

28. A method according to claim 26, wherein the step of positioning at least one intermediate support member between the proximal reception portion of the bone anchor and the support flange of the fixation member includes the step of placing the at least one intermediate support member between the tissue and the reception portion of the bone screw.

29. A method according to claim 26, wherein the step of positioning at least one intermediate support member between the proximal reception portion of the bone anchor and the support flange of the fixation member includes the step of placing the at least one intermediate support member between the tissue and the support flange of the fixation member.

30. A method according to claim 26, wherein the step of positioning at least one intermediate support member between the proximal reception portion of the bone anchor and the support flange of the fixation member includes the steps of placing a first intermediate support member between the tissue and the support flange of the fixation member and placing a second intermediate support member between the tissue and the reception portion of the bone anchor.

31. A method according to claim 27, further comprising the steps of:
a) coupling the at least one intermediate support member and the tissue; and
b) aligning the at least one intermediate support member to the bone to allow subsequent attachment of the tissue.
